(19) United States
(12) Patent Application Publication
(10) Pub. No.: US 2009/0025725 A1
(43) Pub. Date: Jan. 29, 2009

(54) TRANSIENT INTERVENTION FOR MODIFYING THE BREATHING OF A PATIENT

(75) Inventor: John E. Remmers, Calgary (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: UTI LIMITED PARTNERSHIP, Calgary (CA)

(21) Appl. No.: 12/180,465

(22) Filed: Jul. 25, 2008

Related U.S. Application Data
(60) Provisional application No. 60/952,084, filed on Jul. 26, 2007.

(51) Int. Cl. A61M 16/00

(52) U.S. Cl. 128/204.23

(57) ABSTRACT

A method for modifying the breathing of a patient while the patient is experiencing sleep apnea, for example Cheyne Stokes breathing. An instantaneous pulmonary ventilation of a patient is monitored. Limit cycle behavior of the instantaneous pulmonary ventilation is identified. A transient intervention is applied to the patient during a phase of a limit cycle of the instantaneous pulmonary ventilation. The transient intervention has an effect on a breathing state of the patient.

### CROSS-REFERENCE TO RELATED APPLICATIONS

This application claims priority under 35 U.S.C. §119(e) from provisional U.S. Patent Application No. 60/952,084, filed Jul. 26, 2007, the contents of which are incorporated herein by reference.

### BACKGROUND

1. Field

This relates to a method and apparatus for providing transient interventions to stabilize a patient's breathing pattern, and, in particular, to a method and apparatus that includes identifying a patient's limit cycle behavior and applying the intervention according to a phase of the limit cycle.

2. Description of the Related Art

Central sleep apnea is a type of sleep-disordered breathing that is characterized by a failure of the sleeping brain to generate regular, rhythmic bursts of neural activity. The resulting cessation of rhythmic breathing, referred to as apnea, represents a disorder of the respiratory control system responsible for regulating the rate and depth of breathing; that is, a disorder of overall pulmonary ventilation. Central sleep apnea should be contrasted with obstructive sleep apnea, where the proximate cause of apnea is obstruction of the pharyngeal airway despite ongoing rhythmic neural outflow to the respiratory muscles. The difference between central sleep apnea and obstructive sleep apnea is clearly established, and the two can share pathophysiological causal features. Obstructive sleep apnea occurs when physical obstruction of the airway passage occurs in the pharynx. Central sleep apnea derives from a disorder in the breathing control systems. While central sleep apnea can occur in a number of clinical settings, it is most commonly observed in association with heart failure or cardiovascular disease.

Cheyne Stokes breathing is a type of sleep disordered breathing in which respiration of the patient waxes and wanes in a smooth crescendo/decrescendo pattern. This is a form of breathing instability and it may be caused by central sleep apnea.

Two chemoreflex feedback loops control breathing and Cheyne Stokes breathing results from increased gain in these feedback loops. One feedback loop, the peripheral chemoreflex involves a CO₂ and O₂ sensor in the carotid artery. High gain of one or both loops or excessive circulatory delays can cause breathing instability. Other causes of central sleep apnea and Cheyne Stokes breathing include circulatory delay and pharyngeal instability. The other chemoreflex loop involves the central chemoreceptor in the brain which senses brain tissue P_{CO2}. The terms P₀₂ and P_{CO2} represent the partial pressures of oxygen and carbon dioxide, respectively, in a patient's blood stream. Brain tissue P_{CO2} is the partial pressure of carbon dioxide gas in the brain. Arterial P₀₂ and arterial P_{CO2} is the partial pressure of oxygen gas and carbon dioxide gas in the arterial blood of a patient.

Theoretical consideration and experimental observations indicate that a control system with a high chemoreflex gain can display two stable states, one where breathing is steady state (i.e., regular rhythm and tidal volume) and one where breathing assumes a limit cycle. As well, both theoretical and empirical observations indicate that transient perturbation can shift the system from one state to the other.

Central sleep apnea is believed to be caused by a high gain feedback oscillation of the respiratory control system. This control system is highly complex in that it has two feedback loops separated by delays which are state dependant. The feedback loops are dependant upon the respiratory stimuli, namely the arterial P_{CO2} and P_{O2}. The delay between ventilatory intake and the corresponding response in concentration of arterial P_{CO2} and P_{O2} values may, in some cases, be as long as 30 seconds or longer. In some cases, a high gain feedback oscillation loop of the respiratory control system may exist with a periodic repeating cycle with a period that is approximately double the length of the delay. In such a high gain feedback oscillation loop, the arterial P_{O2} may be increasing during a period when no breathing is occurring, and the arterial P_{CO2} may be increasing during a period of hyperventilation. As a result, the system will oscillate in a stable limit cycle behavior without achieving a steady rhythmic breathing pattern that has minimal changes in ventilation and arterial P_{CO2} and P_{O2} values.

### SUMMARY

The high oscillation characteristic of central sleep apnea can be usefully viewed as a stable limit cycle in which ventilation and arterial blood gases oscillate in a predictable pattern in relation to each other. A transient intervention is used to convert the oscillatory limit cycle behavior to a non-oscillatory fixed point behavior.

In one embodiment, there is provided a method for modifying the breathing of a patient. An instantaneous pulmonary ventilation of a patient is monitored. Limit cycle behavior of the instantaneous pulmonary ventilation is identified. The limit cycle behavior of the instantaneous pulmonary ventilation corresponds to a limit cycle having phases. A transient intervention is applied to the patient according to a phase within the limit cycle. The transient intervention has an effect on a breathing state of the patient.

In another embodiment, there is provided an apparatus for treating a breathing disorder. The apparatus comprises a pulmonary ventilation sensor, a transient intervention provider and a controller responsive to signals from the pulmonary ventilation sensor to cause the transient intervention provider to apply a transient intervention to a patient.

These and other objects, features, and characteristics of the device and method, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the device and method. As used in the specification and in the claims, the singular form of “a”, “an”, and “the” include plural referents unless the context clearly dictates otherwise

### BRIEF DESCRIPTION OF THE FIGURES

FIG. 1 shows a block diagram of a method for applying a transient intervention to a patient;

FIG. 2 shows a block diagram of another embodiment of a method for applying a transient intervention to a patient;

FIG. 3 shows a limit cycle characteristic of central sleep apnea with oscillations of arterial P_{CO2} and P_{O2};

FIG. 4 shows a limit cycle characteristic of central sleep apnea with oscillations of brain P_{CO2} and arterial P_{O2}; and

FIG. 5 shows apparatus for carrying out method steps shown in FIG. 1.

### DETAILED DESCRIPTION OF THE EXEMPLARY EMBODIMENTS

In the claims, the word “comprising” is used in its inclusive sense and does not exclude other elements being present. The indefinite article “a” before a claim feature does not exclude more than one of the feature being present. Each one of the individual features described here may be used in one or more embodiments and is not, by virtue only of being described here, to be construed as essential to all embodiments as defined by the claims. The phrase “instantaneous pulmonary ventilation” in this document refers to the ratio of tidal volume divided by respiratory period of a human being's breathing.

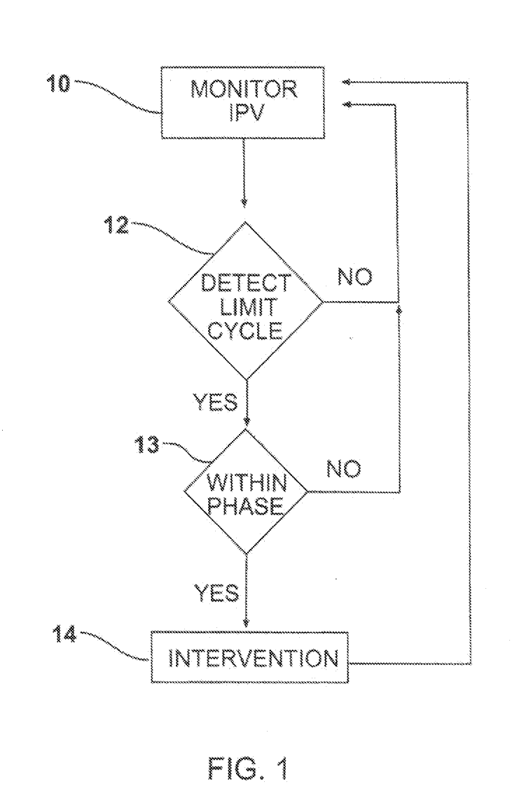

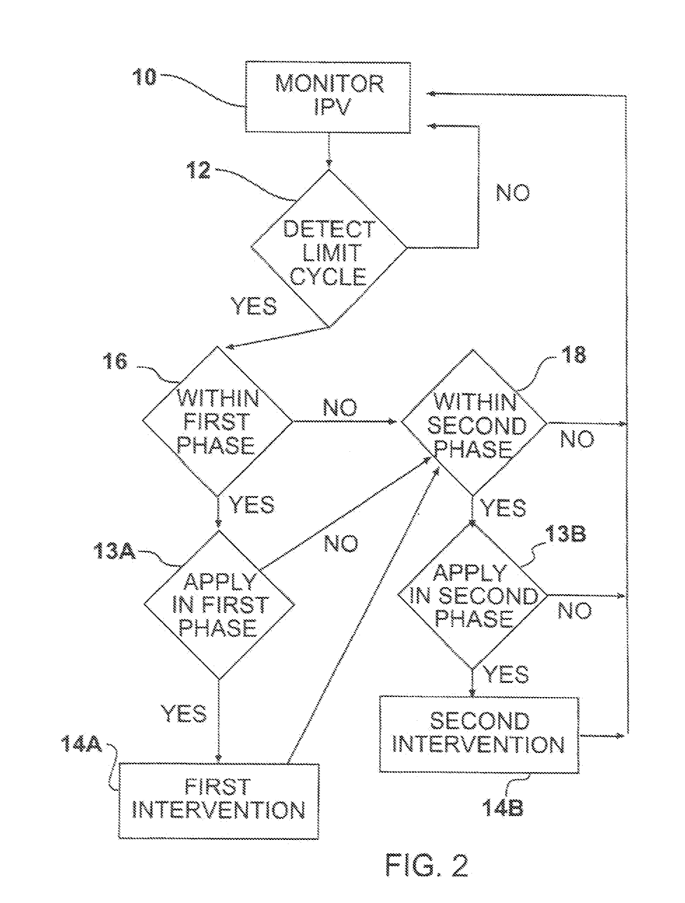

FIGS. 1 and 2 show methods for modifying the breathing of a patient. At step 10 an instantaneous pulmonary ventilation (IPV) of a patient is monitored. As shown at step 12, limit cycle behavior is detected and when, as shown at step 13, the instantaneous pulmonary ventilation is within a phase of the limit cycle, then at step 14 a transient intervention is applied to the patient which affects the alveolar ventilation of the patient. As shown at steps 13A and 13B of FIG. 2, the transient intervention may be applied in a first phase of the limit cycle or a second phase of the limit cycle, or, in some cases, the transient intervention may be applied in both the first and second phases within the limit cycle. For example, the first phase of the limit cycle may correspond to a phase of the limit cycle wherein the instantaneous pulmonary ventilation is a decreasing or minimal. The second phase of the limit cycle may correspond to a phase of the limit cycle wherein the instantaneous pulmonary ventilation is increasing or maximal.

If, as shown in step 16, the limit cycle is within the first phase, and if, as shown in step 13A, a transient intervention is to be applied during the first phase, then at step 14A a first transient intervention is applied to the patient. If, as shown in step 18, the limit cycle is within the second phase, and if, as shown in step 13B, a transient intervention is to be applied during the second phase, then at step 14B a second transient intervention may be applied to the patient. If the first phase of the limit cycle corresponds to the instantaneous pulmonary ventilation decreasing or minimal, then the first transient intervention may be an excitatory transient intervention. If the second phase of the limit cycle corresponds to the instantaneous pulmonary ventilation increasing or maximal, then the second transient intervention may be a mitigating transient intervention. The monitoring step 10 may be carried out continuously or intermittently.

Step 12 of detecting limit cycle behavior may be carried out by any of several methods. A threshold range may be determined experimentally so that limit cycle behavior is detected when the instantaneous pulmonary ventilation is outside of the threshold range. The threshold range is selected so that when a patient is breathing normally, the instantaneous pulmonary ventilation lies within the threshold range. As well, the temporal pattern of ventilation can be monitored and the intervention can be timed to occur at particular points in the limit cycle. For example, a normal range of the instantaneous pulmonary ventilation may be determined for the patient, and the occurrence of a measured instantaneous pulmonary ventilation outside of this range may be considered to be in excess of the threshold.

A single measurement outside of the normal range may be flagged for potential threshold crossing, but a transient intervention is only applied when a further measurement that is also outside of the normal range occurs within a set period of time. In a further example, the occurrence of successive instantaneous pulmonary ventilation measurements beyond a threshold may be used to trigger a transient intervention. In a still further example, a number of successively increasing instantaneous pulmonary ventilation measurements may be considered to cross a threshold either based on the number of successive increasing measurements or the magnitude of the increase from measurement to measurement. For example, a threshold may be considered to have been crossed when successive differences between instantaneous pulmonary ventilation measurements are each above a threshold difference. Hence, the threshold may effectively be a magnitude or a rate of change of the instantaneous pulmonary ventilation that is sustained over a pre-determined number of breaths or a pre-determined period of time. The limit cycle behavior may also be detected by autocorrelation, which compares repeating patterns of the instantaneous pulmonary ventilation over time. The limit cycle behavior may be detected by using Poincaré plots, which compares consecutive breaths of the patient. The limit cycle behavior may also be detected by detecting limit cycle behavior in other cycles that are correlated to the instantaneous pulmonary ventilation. For example, limit cycle behavior in arterial P_{CO2} and arterial P_{O2} may indicate corresponding limit cycle behavior in instantaneous pulmonary ventilation.

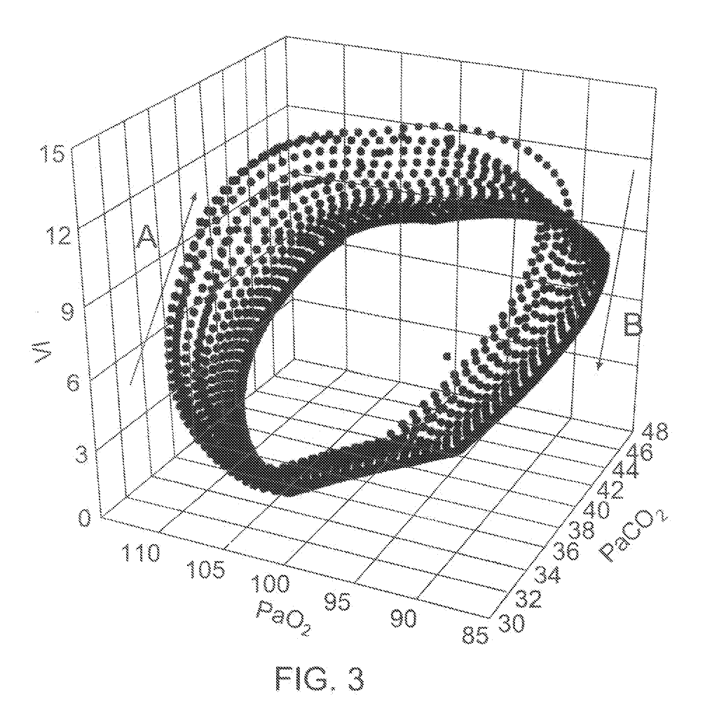

FIG. 3 shows a typical limit cycle in which ventilation and arterial blood gases oscillate in a predictable pattern in relation to each other. A pattern is formed by oscillations in the ventilation, arterial P_{O2} and P_{CO2} associated with a stable limit cycle behavior of the respiratory control system. In particular, the instantaneous pulmonary ventilation V considered on its own oscillates in a stable limit cycle within the stable limit cycle of the respiratory control system. The system shown started to oscillate as a result of three fold increase in the chemosensitivity of the central chemoreflex loop with a normal value of the cardiac output (6.2 l/mins). The data is derived from a realistic simulation of the respiratory control system. A stable non-oscillatory fixed point solution (not shown) co-exists as an alternative to the limit cycle solution in Cheyne Stokes breathing.

To improve the breathing of the patient the amplitude of the oscillations of the instantaneous pulmonary ventilation is reduced and the oscillations are reduced to a non-oscillatory fixed point behavior. Reduction of the oscillatory behavior of the instantaneous pulmonary ventilation has the effect of reducing the limit cycle behavior of the respiratory control system. The oscillatory limit cycle of the instantaneous pulmonary ventilation may be a component of the oscillatory limit cycle of the respiratory control system.

A plurality of transient interventions may be applied within the oscillatory limit cycle of the instantaneous pulmonary ventilation. For example, the plurality of transient interventions may be applied periodically within phases of the limit cycle. A mitigating transient intervention that has the effect of reducing alveolar hyperventilation and mitigating the gas exchange consequences of alveolar hyperventilation may be applied periodically within a phase of the cycle when the instantaneous pulmonary ventilation is increasing or maximal. An excitatory transient intervention that has the effect of augmenting alveolar ventilation may be applied periodically within a phase of the cycle when the instantaneous pulmonary ventilation is decreasing or minimal. As periodic interventions are applied it may be beneficial to observe any shifts in the oscillatory limit cycle. The intensity of the applied transient interventions may be adjusted in response to the observed shift in the oscillatory limit cycle.

If each one of the plurality of transient interventions are applied consecutively, it may be beneficial to adjust the time between the consecutive applications of the plurality of transient interventions in response to the observed shift in the oscillatory limit cycle. For example, if the oscillatory limit cycle decreases as the plurality of interventions are applied, then the transient interventions may be adjusted so that the intensity of each of the interventions decreases as the oscillatory limit cycle decreases. Because of memory in the control system, a regimen of a sequence of transient interventions of predetermined magnitude at predetermined times in the cycle may result in the conversion of limit cycle to steady state behavior with lower transient intervention intensity, thereby lessening the chance of an arousal.

Before each transient intervention is applied, the limit cycle behavior of the respiratory control system as a whole may be quantified. In another approach, the limit cycle behavior of only the instantaneous pulmonary ventilation component of the respiratory control system may be quantified. Additionally, precise times within the limit cycle may be determined so that when a transient intervention is applied, the result will be a conversion of the instantaneous pulmonary ventilation from limit cycle to fixed point behavior.

The limit cycle of the respiratory control system can be quantified by plotting instantaneous pulmonary ventilation versus oxygen saturation of arterial blood. For a transient intervention that impairs pulmonary gas exchange, and thereby mitigates the effect of alveolar hyperventilation, application during the ascending phase A (FIG. 3) of the limit cycle may be appropriate. The ascending phase corresponds to a rapid increase in the instantaneous pulmonary ventilation of the patient. For a transient intervention that assists pulmonary gas exchange, and thereby augments alveolar ventilation, application during the descending phase B (FIG. 3) may be appropriate. The descending phase corresponds to a rapid decease in the instantaneous pulmonary ventilation of the patient. After application of the intervention the succeeding limit may be compared to the pre-intervention cycles to access the effect of the transient intervention. If a complete conversion to the fixed point behavior is not observed, the intervention may be repeated on succeeding cycles with timing and intensity of these applications being guided by the observed shift in limit cycle caused by the preceding intervention.

In FIG. 3, the flat, minimal phase of the graph between the ascending phase A and the descending phase B corresponds to an instantaneous pulmonary ventilation is minimal. The instantaneous pulmonary ventilation will be minimal when the tidal volume of the patient is minimal. For example, the tidal volume of the patient will be minimal if the patient ceases breathing. The nearly flat, maximal phase of the graph between the descending phase B and the ascending phase A corresponds to maximal instantaneous pulmonary ventilation. Maximal instantaneous pulmonary ventilation is caused by high tidal volume breathing of the patient. For example, high tidal volume breathing of the patient may correspond to alveolar hyperventilation.

More accurate timing of the intervention may be achieved by employing a realistic computational model of the respiratory control system. A more realistic computational model would allow development of an adaptive controller guided by the predictions derived from the embedded model. Critical parameters reflecting the patient's condition may be introduced into the model and delays between gas exchange in the lungs and detection of chemical stimuli by peripheral and central chemoreceptors may be calculated. A respiratory chemoreflex simulator may be used to identify regimes of external transient interventions applied to a respiratory control system in limit cycle behavior. An adaptive control system can monitor the results of the intervention and apply further interventions and regimes as suggested by predictions of the simulator. A computational model may be used to identify the opportune times and optimal regimens of transient interventions that can most readily convert the system from a limit cycle to a steady state. The computational model may interact with a larger controlling program which identifies the behavior of the model and systematically introduces increase or decrease in alveolar ventilation for various durations and various times in the limit cycle.

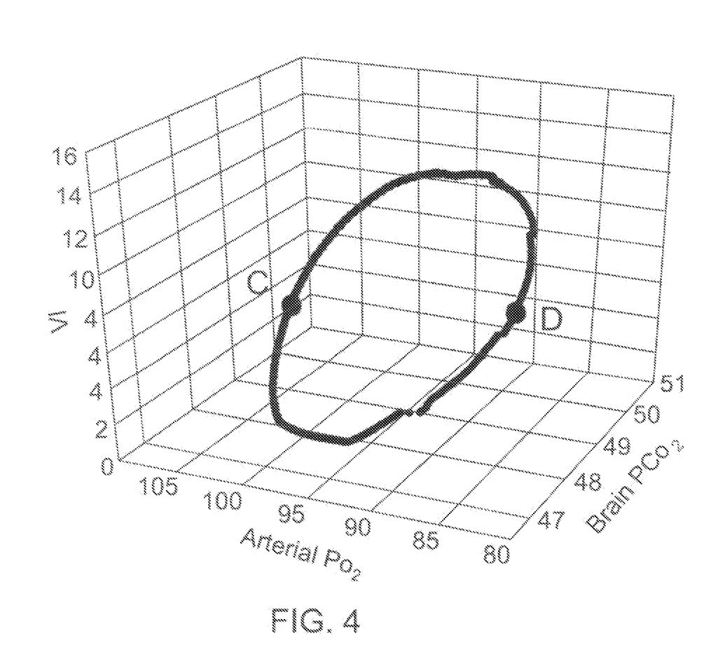

As shown in FIG. 4, the model may then calculate brain tissue P_{CO2} and display the limit cycle in the new set of coordinates with ventilation V, arterial P_{O2} and brain P_{CO2} associated with a stable limit cycle behavior of the respiratory control system. In FIG. 4, as in FIG. 3, the system started to oscillate as a result of a three fold increase in the chemosensitivity of the central chemoreflex loop with normal value of the cardiac output (6.2 l/min). Tracing the value of the instantaneous pulmonary ventilation, the controller will engage the mitigating, or dampening, transient intervention at the middle point of the ascending phase of the limit cycle C. The middle point of the ascending phase C corresponds to a zero-crossing of the instantaneous pulmonary ventilation. The zero-crossing corresponds to the instantaneous pulmonary ventilation changing from lower than average instantaneous pulmonary ventilation to higher than average instantaneous pulmonary ventilation. The average instantaneous pulmonary ventilation may be the average instantaneous pulmonary ventilation over the previous cycle. The zero-crossing may correspond generally to a maximal rate of change of the instantaneous pulmonary ventilation. The optimal length of the applied intervention, which will generate a complete conversion to the fixed point behavior may be determined by results from the computer simulation.

A similar strategy may be employed in the case of an excitatory transient intervention by applying the intervention at the middle point of the descending phase of the limit cycle D. The middle point of the descending phase D corresponds to a zero-crossing of the instantaneous pulmonary ventilation. The zero-crossing corresponds to the instantaneous pulmonary ventilation changing from higher than average instantaneous pulmonary ventilation to lower than average pulmonary ventilation. The zero-crossing may correspond generally to a maximal rate of change of the instantaneous pulmonary ventilation. Again, the computer simulation may be performed to determine the optimal length of the intervention. In this example, the middle point of the ascending phase C and the middle point of the descending phase D each correspond generally to an extreme value of P_{O2} concentration. The ascending phase C corresponds to maximal arterial P_{O2} concentration and the descending phase D corresponds to minimal arterial P_{O2} concentration.

The transient interventions may be either respiratory or non-respiratory in nature. In the respiratory intervention, a ventilatory assist or an impairment of gas exchange can be transiently imposed at a particular point in the limit cycle and applied intervention will either transiently decrease the amplitude of the oscillations or completely convert the behavior of the system to the fixed point. Once conversion from the limit cycle to a fixed point behavior has occurred, the system will generally remain stable in the non-oscillatory steady state for a prolonged period of time.

Some examples of transient interventions will now be described. Generally, a transient intervention is any intervention that may be applied to a patient that affects the breathing state of the patient. The examples of transient interventions discussed below are known in the art and need not be described in detail.

The transient intervention may be an electrical stimulus applied to the patient. The electrical stimulus may be applied to the upper airway muscles, the hypoglossal nerve, the vagus nerve or other excitable tissue for the treatment of sleep apnea. The electrical stimulus may also be applied to other muscle groups of the patient. For example, the electrical stimulus may be applied to the shoulder, neck, arm, leg or other suitable muscle. The electrical stimulus may mimic movement of the shoulder, neck, arm, leg or other suitable muscle of the patient. The stimulus may stimulate neural afferent fibers that are normally activated during muscular exercise and stimulate breathing. An electrical stimulus may be applied to the patient's skin to provide a pinching, stinging or shocking sensation.

The transient intervention may be applied by a manual device provided to act on the patient. The manual device may cause passive movement of the patient's limbs or muscles. The passive movement may be movement of the limb joints of the patient or may be compression of the muscles. For example, manual movement of the patient's feet at the ankle may increase alveolar ventilation of the patient. The manual device may provide rhythmic stimulation of the patient. For example, the manual device may be a rocking bed or a rhythmically moving stuffed animal for a child patient. Movement of parts of the patient's body will often entrain the breathing of the patient into a corresponding rhythm. Passive motion of the limbs of the patient will often induce increased ventilation. A human being, such as a care provider or bed partner, may replace the manual device by manually moving the patient. Pneumatic compression of the lower limbs can also be used to activate neural afferents that stimulate breathing.

The transient intervention may cause manipulation of the air flow or airway passages of the patient. For example, the transient intervention may be the application of continuous positive airway pressure (CPAP) to the patient. The intervention may also be the application of controlled re-breathing or low flow CPAP to the patient. A transient period of rebreathing caused by an external dead space may reduce alveolar ventilation. For example, when the instantaneous pulmonary ventilation is increasing, rebreathed air may be supplied to the patient and when the instantaneous pulmonary ventilation is decreasing atmospheric or oxygen rich air may be supplied to the patient. The transient intervention may be a mandibular protrusion device.

The transient intervention may be auditory signals. The auditory signals may be verbal commands made by a human being or an auditory device. The auditory device may give instructions to the patient while the patient is sleeping.

Generally, the transient intervention should be applied so that the patient does not awake because of the transient intervention. It may also be beneficial to use a sensor to detect when the patient is asleep so that the intervention is not applied when the patient is awake and thereby cause irritation to the patient. An important advantage of the transient intervention technology is that interventions that may be somewhat noxious if applied repetitively may be well tolerated if applied transiently and in isolation. Nonetheless, these transient interventions may cause a long term conversion of breathing from limit cycle to steady state and, thereby, provide effective and feasible therapy.

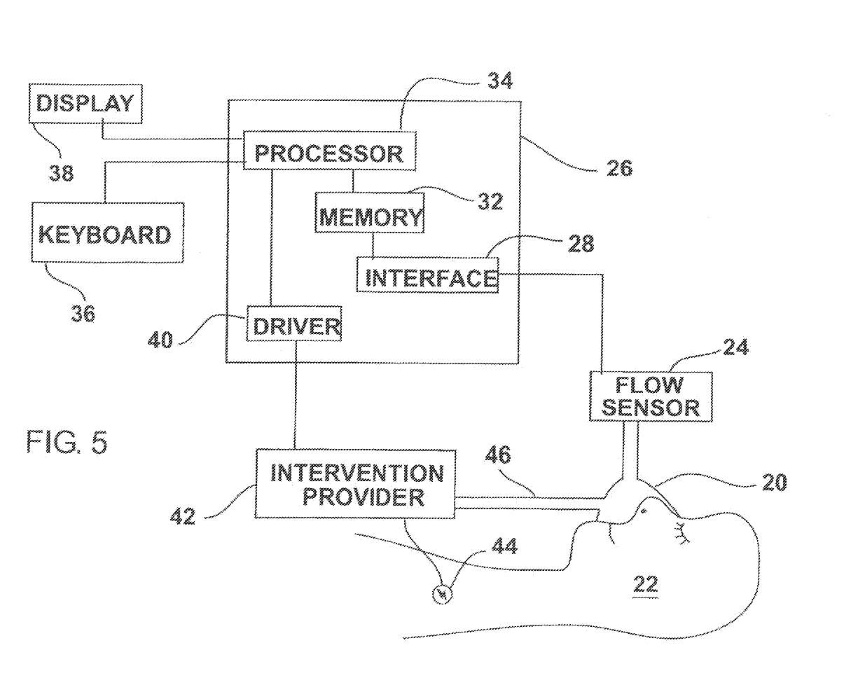

Various apparatus may be used to carrying out the method described in the claims. In one example shown in FIG. 5, a mask 20 is attached a patient 22 and the mask 20 fitted with a flow sensor 24. The flow sensor 24 detects pulmonary ventilation. The flow sensor 24 is part of a means for monitoring an instantaneous pulmonary ventilation of a patient. Pulmonary ventilation or other respiration characteristic may be monitored by any suitable device such as a device capable of detecting flow directly or indirectly, such as a pressure sensor, pneumotacograph or ultrasonic sensor. Other sensors may be used to monitor the instantaneous pulmonary ventilation or breathing characteristic of the patient. A signal from the flow sensor 24 is provided to a controller 26 through a sensor interface 28. The sensor interface 28 outputs signals to a memory 32 within the controller 26. The memory 32 is sampled by processor 34. Processor 34 carries out the monitoring of instantaneous pulmonary ventilation and detection of limit cycle behavior of the instantaneous pulmonary ventilation through, for example, software or firmware. Processor 34 may be any suitable electronic processor 34 such as a chip or chip in a general purpose computer or an application specific chip that is programmed or otherwise configured to carry out the method steps described here.

The processor 34 is connected to conventional input devices, such as a keyboard 36 and to output devices such as a display 38. The processor 34 is also in this example connected to a driver 40 for a transient intervention provider 42. The transient intervention provider 42 may for example be connected to the patient via electrodes 44 or a breathing tube 46 or other suitable transient intervention applicator. The driver 40 may be a stand alone device or may be embedded in hardware, firmware or software in the processor 34. In some embodiments, the interface 28, memory 32, processor 34 and driver 40 may be carried on a monolithic semi-conductor device.

Although the invention has been described in detail for the purpose of illustration based on what is currently considered to be the most practical and preferred embodiments, it is to be understood that such detail is solely for that purpose and that the device and method are not limited to the disclosed embodiments, but, on the contrary, is intended to cover modifications and equivalent arrangements that are within the spirit and scope of the appended claims. For example, it is to be understood that the method and device contemplate that, to the extent possible, one or more features of any embodiment can be combined with one or more features of any other embodiment.

What is claimed is:

1. A method for modifying the breathing of a patient, comprising the following steps:
monitoring an instantaneous pulmonary ventilation of a patient;
identifying limit cycle behavior of the instantaneous pulmonary ventilation, the limit cycle behavior corresponding to a limit cycle having phases; and
applying a transient intervention to the patient according to a phase of the limit cycle, the transient intervention having an effect on a breathing state of the patient.

2. The method of claim 1, wherein applying the transient intervention further comprises applying an excitatory transient intervention in a first phase of the limit cycle.

3. The method of claim 2, wherein applying the transient intervention further comprises applying a mitigating transient intervention in a second phase of the limit cycle.

4. The method of claim 2, wherein the first phase of the limit cycle corresponds to decreasing instantaneous pulmonary ventilation or minimal instantaneous pulmonary ventilation within the limit cycle.

5. The method of claim 3, wherein the second phase of the limit cycle corresponds to increasing instantaneous pulmonary ventilation or maximal instantaneous pulmonary ventilation within the limit cycle.

6. The method of claim 1, wherein the step of applying the transient intervention further comprises applying a plurality of transient interventions within the limit cycle; and the plurality of transient interventions are applied periodically within the phase of the limit cycle.

7. The method of claim 6, wherein applying the plurality of transient interventions further comprises the transient interventions being periodically applied when the instantaneous pulmonary ventilation is increasing or maximal.

8. The method of claim 6, wherein applying the plurality of transient interventions further comprises the transient interventions being periodically applied when the instantaneous pulmonary ventilation is decreasing or minimal.

9. The method of claim 6, wherein each transient intervention of the plurality of transient interventions have an intensity, and further comprising the steps of:
observing a shift in the limit cycle as the plurality of transient interventions are applied; and
modifying the intensity of each transient intervention of the plurality of transient interventions in response to the observed shift in the limit cycle of the instantaneous pulmonary ventilation.

10. The method of claim 9, wherein the plurality of transient interventions are applied consecutively, and consecutive applications of the plurality of transient interventions have a time between them, and further comprising the step of modifying the time between the consecutive applications of the plurality of transient interventions in response to the observed shift in the limit cycle of the instantaneous pulmonary ventilation.

11. The method of claim 9, wherein the limit cycle decreases as the plurality of the interventions are applied; and the step of modifying each transient intervention comprises decreasing the intensity of the plurality of interventions as the limit cycle decreases.

12. The method of claim 6, wherein the limit cycle has the phase of the limit cycle corresponding to increasing instantaneous pulmonary ventilation; and the plurality of transient interventions are applied at a zero-crossing within the increasing phase.

13. The method of claim 6, wherein the limit cycle has the phase of the limit cycle corresponding to decreasing instantaneous pulmonary ventilation; and the plurality of transient interventions are applied at a zero-crossing within the decreasing phase.

14. The method of claim 10, further comprising the step of predicting the optimal time between the consecutive applications of the plurality of transient interventions and the optimal intensity of each of the transient interventions based on an embedded model before the steps of modifying the time between the consecutive applications of the plurality of transient interventions and modifying the intensity of each of the transient interventions.

15. The method of claim 14, further comprising providing an adaptive controller to perform the steps of modifying the time between the consecutive applications of the plurality of transient interventions and modifying the intensity of each of the transient interventions.

16. The method of claim 1, further comprising the step of monitoring an oxygen saturation of the arterial blood of the patient and in which the step of applying the transient intervention when the instantaneous pulmonary ventilation is changing further comprises applying the transient intervention when the oxygen saturation of the arterial blood of the patient is at an extreme value.

17. An apparatus for treating a breathing disorder, comprising:
a sensor adapted to detect a characteristic of respiration;
a transient intervention system; and
a controller adapted to control operation of the transient intervention system responsive to signals from the sensor so as to cause the transient intervention system to apply a transient intervention to a patient.

18. The apparatus of claim 17, wherein the controller is configured to:
monitor an instantaneous ventilation of a patient;
detect limit cycle behavior of the instantaneous ventilation, the limit cycle behavior corresponding to a limit cycle having phases; and
activate the transient intervention system within a phase of the limit cycle.

19. The apparatus of claim 18, wherein the controller is configured to activate the transient intervention system to provide a mitigating intervention within a first phase of the limit cycle.

20. The apparatus of claim 18, wherein the controller is configured to activate the transient intervention system to provide an excitatory intervention within a second phase of the limit cycle.
